(19) United States
(12) Patent Application Publication
(10) Pub. No.: US 2026/0345415 A1
(43) Pub. Date: Nov. 5, 2026

(54) SUPPORT DEVICE OR SYSTEM FOR POSITIONING AN INFANT OR A PATIENT FOR A LUMBAR PUNCTURE OR OTHER MEDICAL OPERATIONS AND USE THEREOF

(71) Applicant: Gateway Paediatrics Inc., Yarmouth (CA)

(72) Inventor: Joel COX, Yarmouth (CA)

(73) Assignee: Gateway Paediatrics Inc., Yarmouth (CA)

(21) Appl. No.: 19/473,743

(22) PCT Filed: Jul. 26, 2024
(86) PCT No.: PCT/CA2024/050993

§ 371 Date: Oct. 8, 2025

Related U.S. Application Data
(60) Provisional application No. 63/516,191, filed on Jul. 28, 2023.

(51) Int. Cl. A61G 15/10; A61B 10/00

(52) U.S. Cl. CPC A61G 15/105; A61B 10/0045; A61B 2010/0077; A61B 2503/04

(57) ABSTRACT

The present invention relates to a support system or device for lumbar punctures; in particular, this invention comprises of a lumbar puncture support system/device for the positioning of the infant that maintains an optimal quality of immobilization of the infant during a lumbar puncture. The device comprises a seat base defining a leg support portion, a hip support portion and a back support portion. The hip support and the back support portion receives a hip of the infant. The leg support portion is positioned higher than the hip support position to cause thighs and knees of the infant to be close to or to touch an upper body of the infant to provide clear and exposed back of the infant for the lumbar puncture.

### CROSS-REFERENCE TO RELATED APPLICATIONS

The present application claims priority to U.S. Provisional Application No. 63/516,191, filed on Jul. 28, 2023, the content of which is incorporated herein by reference in its entirety.

### BACKGROUND OF THE INVENTION

### Field of the Invention

The present invention relates to a support system or device for a lumbar puncture, in particular, a lumbar puncture support system/device for pediatric patients or infants.

### Description of Related Art

In the course of routine medical care for infants, it may be necessary to conduct a procedure called “lumbar puncture” which involves the insertion of a needle into the base of a spinal column (between two bones in the lumbar spine (L1 to L5)) in order to extract fluid for diagnostic purposes. The targeted area to access this fluid is approx. 3-5 mm in diameter.

Performing successful lumbar punctures is a challenge to both patients/infants and health-care professionals. Currently, failure rates for lumbar puncture (to infants) are as high as 50%.

Many factors, including the following, may contribute to the success or failure of this procedure:
1) The clinical condition of the infant;
2) The management of pain related to the extent of patient movement;
3) The experience, technique and confidence of the operator conducting the procedure; and,
4) The experience of the assistant positioning the baby.

In a traditional lumbar puncture, an infant/patient would be positioned in a side-lying position as shown in FIG. 1, or would be positioned in a sitting-up position as shown in FIGS. 2 and 3.

The side-lying position is the most common practice as the patient seems to be more stable; however, the alignment of the spine is often disrupted/inconsistent/unideal and creates additional challenges to the success of the procedure. On the other hand, the sitting position is associated with better outcomes but is often avoided as the holding technique is more complex. The traditional positionings or methods does not appropriately address the positioning of the infant in an optimal way that maintains a high quality of immobilization of the infant during a lumbar puncture.

Various attempts were made to address one or more of the issues shown above. For example, Chinese Patent No. 110559149B and Chinese Utility Model Nos. 212997262(U) and 218338668(U) provide devices for placing a patient in a side-lying position, each case detailing a bed and/or device where a patient would lie on his/her side with their knees brought closer to his/her chest/upper body to round and expose the infant's back for the operation, and various means are provided to securely immobilizing the patient's body. While these attempted to provide more stable means for the operation, they do not fundamentally solve the issue of the frequent misalignment/disruption of alignment of the spine as stated above.

Various other attempts are also made to place a patient in a sitting position for lumbar punctures. For example, French Patent Application Publication No. 3095121 shows a chair for lumbar puncture, where a patient would sit and hug the front support of the chair to expose the back for the operation. US Patent Application Publication Nos. 20070272254A1 and 20230200560A1 disclose a device for positioning a patient for a lumbar puncture, where a patient would face to and hug the device such that the back of the patient would be rounded and exposed for the operation. While these solutions may be adapted or modified for use on an infant; their structures do not allow natural curling of the spine to provide a more clear and effective exposure for a lumbar puncture.

Accordingly, there has been a growing demand for a system, i.e., tool or device, to address these shortcomings and to improve the success rate for lumbar punctures for infants.

### BRIEF SUMMARY OF THE INVENTION

According to an object of the present invention, the present invention provides a system, device and tool that improves the positioning of the infant while maintaining an improved quality of immobilization during a lumbar puncture.

According to another object of the present invention, it provides an infant/child seating device that offers physicians/operators a better positioning solution that improves the stability, comfort, and safety of the infant during the lumbar puncture and increases the rate of success.

According to a preferred embodiment of the present invention, it provides a device for positioning an infant for a lumbar puncture, comprising: a seat base defining a leg support portion, a hip support portion and a back support portion, wherein the hip support and the back support portion receives a hip of the infant, and the leg support portion positioned higher than the hip support position, causing thighs and knees of the infant to be close to or to touch an upper body of the infant to expose a back of the infant for the lumbar puncture.

### BRIEF DESCRIPTION OF THE SEVERAL VIEWS OF THE DRAWING

The present invention is now described in more detail with reference to the accompanying drawings in which:

FIGS. 1 to 3 show conventional positionings of infant for lumbar punctures;

FIG. 4 is a perspective view of a first embodiment of the support system for a lumbar puncture according to the present invention;

FIG. 5 is a rear view thereof;

FIG. 6 is an enlarged fragmentary view thereof;

FIG. 7 is another rear view thereof with an infant positioned on the system;

FIG. 8 is a side view thereof;

FIG. 9 is another side view thereof with an infant positioned on the system;

FIG. 10 is a perspective view of a second embodiment of the support system for a lumbar puncture according to the present invention;

FIG. 11 is a rear view thereof;

FIG. 12 is a side perspective view thereof;

FIG. 13a is a side view thereof;

FIG. 13b is a front view of the base thereof;

FIG. 13c is a cross-sectional right-side view of the base thereof at D-D shown in FIG. 13b;

FIG. 14 is another perspective view thereof; and

FIG. 15 is a rear perspective view thereof.

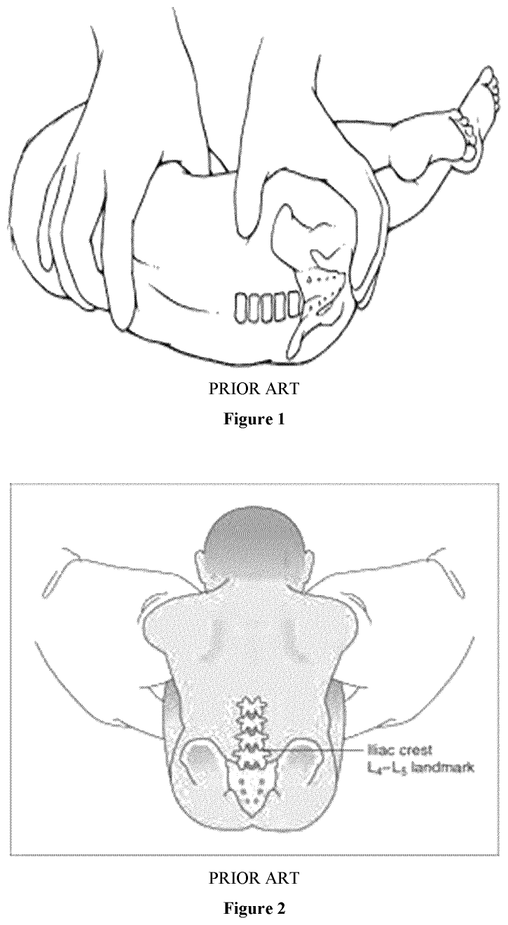

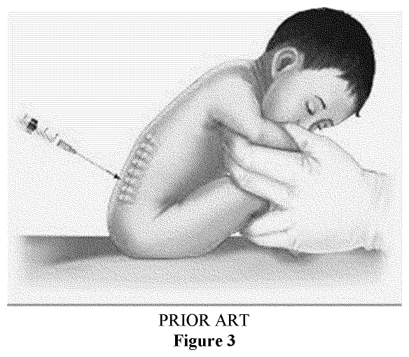

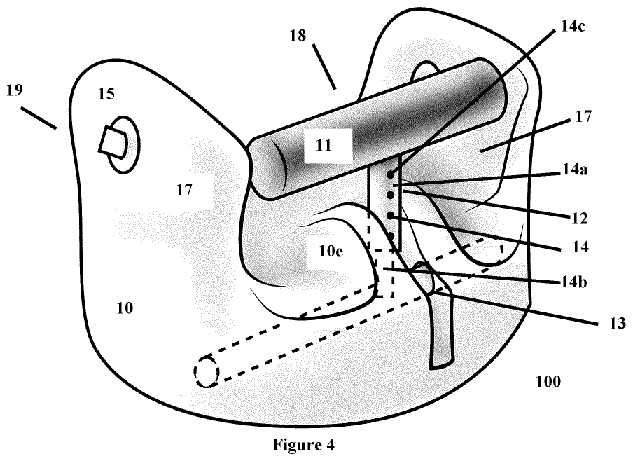

### DETAILED DESCRIPTION OF THE INVENTION

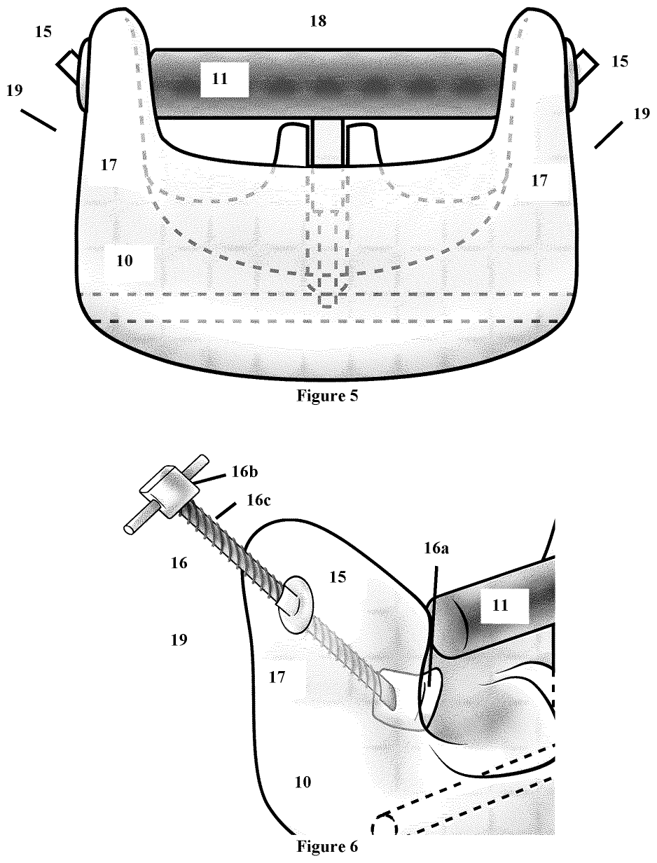

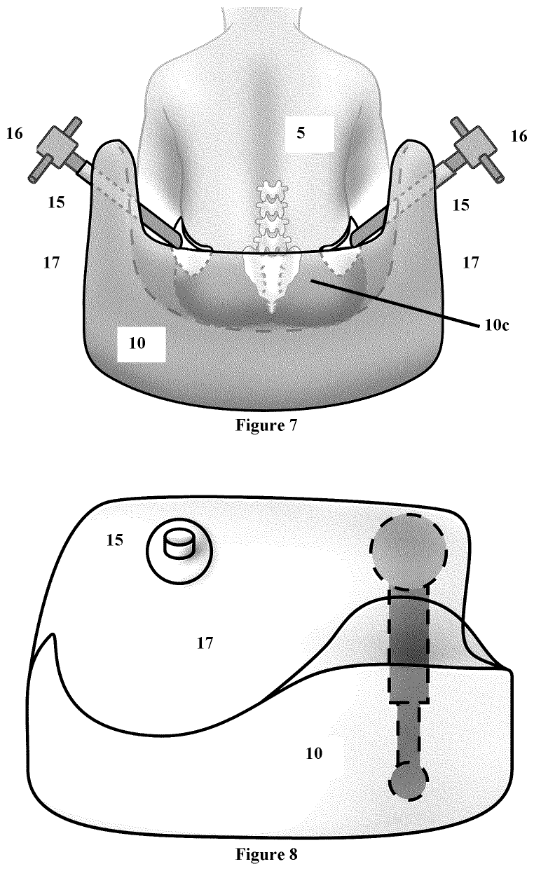

FIGS. 4 to 9 show the first embodiment of the support system 100 for a lumbar puncture according to the present invention.

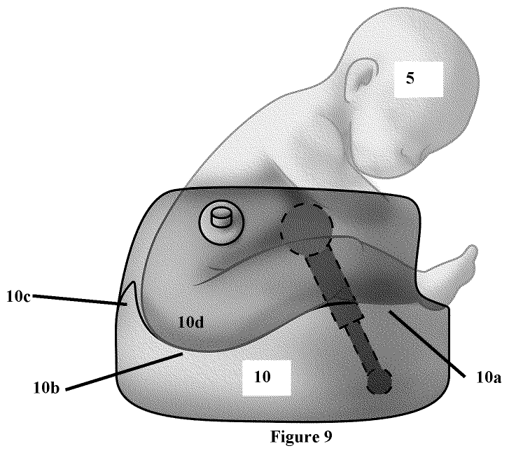

The support system 100 comprises a base 10 for an infant 5 to be positioned. As shown in FIG. 9, from the side view, the base 10 interfaces with the lower body of the infant 5, defining a leg support portion 10a, hip support portion 10b, and back support portion 10c. The leg support portion 10a and hip support portion 10b are arranged/positioned such that the thighs of the infant 5 would be positioned higher than the hip portion of the infant 5 thus the thighs/knees would be drawn closer to or would touch the chest/upper body of the infant 5. The hip support portion 10b and the back support portion 10c define a dip/pocket 10d to receive /hold the hip of the infant 5, such that infant's back is slightly curled and is clearly and stably exposed for the lumbar puncture.

To promote the bending position of the infant 5, the support system 100 further comprises a body immobilizer 18 for immobilizing the bending body position/posture of the infant 5. The body immobilizer 18 comprises a vertical bar 12, which is pivotably attached to the base 10 through pivotable means (or horizontal bar) 13 disposed at one end of the vertical var 12, and an interfacing horizontal bar 11, which is disposed at the other distal end of the vertical bar 12. The interfacing horizontal bar 11 interfaces with/about upper abdominal/lower breast portion of the infant 5 and hold down the thighs above the knees against the base 10 to promote bending position/posture to expose the lower back of the infant 5 as shown in FIG. 9. The height of the interfacing horizontal bar 11 can be adjusted along a vertical bar 12. The vertical bar 12 is telescopically extendible, and the height (or its length) may be releasable adjusted using the height adjustment means 14. For example, the height adjustment means 14 comprises a first bar member 14a and a second bar member 14b being telescopically/slidably engaged each other. The first bar member 14a defines a plurality of predetermined positions or through-holes 14c, and the second bar member 14b comprises a releasable latch that engages with a desirable one of the positions 14c to provide a desirable height position. Various other methods/means may be provided for implementing substantially the same or similar features.

In order to effectively manage movements of the infant's legs, the support system 100 further comprises a leg dividing member 10e to open legs and to individually restrict movement of each leg.

Referring to FIG. 6, to further promote the bending position of the infant 5, the support system 100 provides a lateral body immobilizer 19 on a lateral wall 17. The lateral body immobilizer 19 is adjustable to accommodate a range of body sizes of the infants. For example, according to one embodiment of the present invention, the lateral body immobilizer 19 comprises an actuator 16, comprising a screw member 16b having a handle 16c. A pad (swivel shoe or movable jaw) 16a is provided at the other end of the screw member 16b, which engages/interfaces with a side of the hip of the infant 5. Ports 15 are disposed on the lateral walls 17 of the base 10, each receives the actuator 16. A user uses the handle 16c to actuate the screw member 16b to adjust the length from the lateral wall 17 to suitably engage the swivel shoe 16a to engage with anterior superior iliac spine of the hip of the infant 5 to immobilize the lower body/pelvis of the infant 5 to minimize the movement/to be stationary. As shown on FIG. 9, the infant 5 leans forward and over the horizontal bar 11, such that the infant's back would be accessibly exposed for a lumbar puncture.

The support system 100 may be made of a suitable material, such as plastic or acrylonitrile butadiene styrene (ABS) and may further be provided with a comfortable padding made of a safe and soft material such as Ethyl Vinyl Acetate (or EVA).

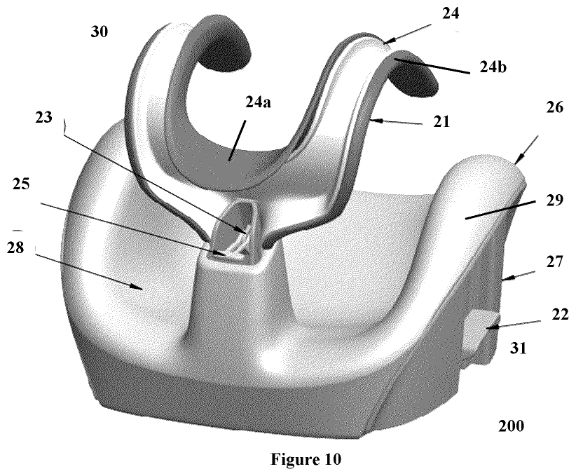

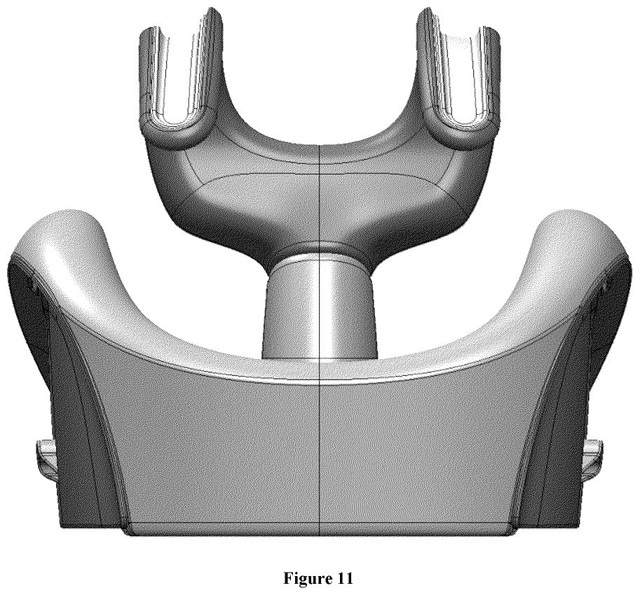

FIGS. 10 to 16 show the second embodiment of the support system 200 for a lumbar puncture according to the present invention. The system 200 comprises a seat base 27. The seat base 27 may be made of a suitable material, such as plastic or acrylonitrile butadiene styrene (ABS). For interfacing with the lower body of the infant 5, a seat foam 26 is provided on the seat base 27. The seat foam 26 may be made of a suitable material, such as ethylene-vinyl acetate (or EVA) foam.

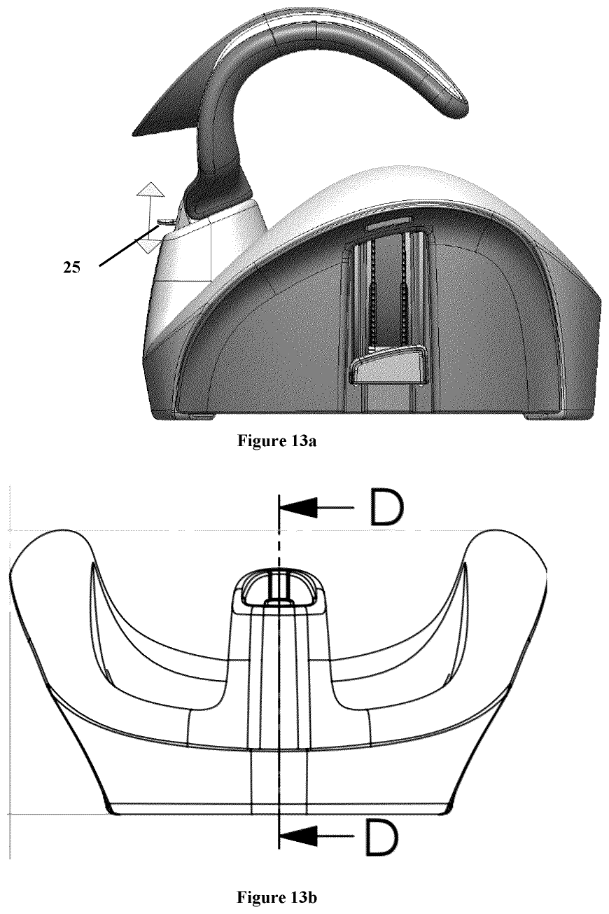

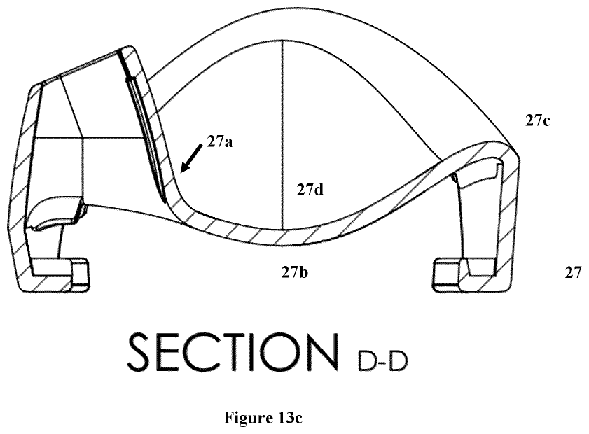

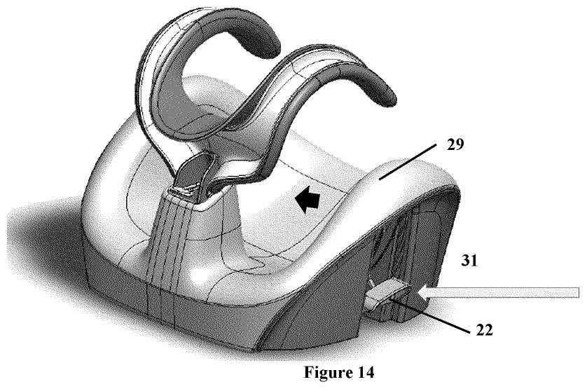

Referring to FIGS. 13b and 13c, like the support system 100, the seat base 27 and the seat foam 26 are shaped such that the portions 27b and 27c of the seat foam 26/seat base 27 interfaces with the hip of the infant 5 defines a dip/pocket 27d to promote the bending of the body at the hip. When the infant 5 is seated, the hip is positioned lower than the back side of thighs of the infant 5 that would be positioned on a leg support portion 27a of the seat foam 26/the seat base 27. This shape of the seat base 27 and the form 26 promotes upper side of the thighs/knees to be close to the chest/upper body of the infant 5 in order to provide better exposure of the lower back of the infant 5 for lumbar punctures.

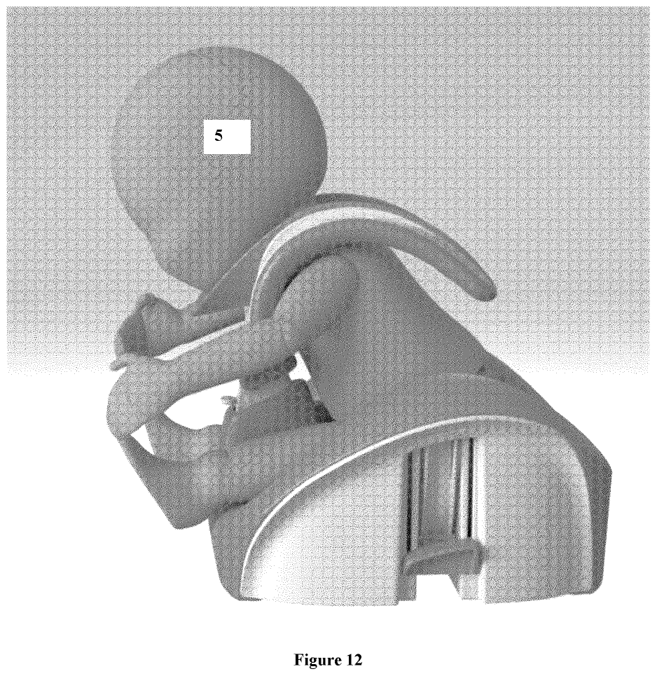

To promote a stable bending position of the infant 5, the support system 200 further comprises a body immobilizer 30 for immobilizing the bending body posture of the infant 5. The body immobilizer 30 comprising a breast brace 24. The breast brace 24 comprises shoulder portions 24b, which are shaped to wrap around and over the shoulders of the infant 5 to prevent or minimize the upward movement of the upper body of the infant 5 during the procedure. The breast brace 24 further defines a chin-rest portion 24a for receiving the chin of the infant 5 as shown in FIG. 12. The seat base 27 comprises a mirror-handle 28 extends upwardly from the front portion of the seat base 27 for receiving the breast brace 24. The breast brace 24 is designed to be slightly tilted forward such that the breast of the infant 5 would be naturally against the breast brace 24, to rest the chin of the infant 5 on the chin-rest portion 24a of the breast brace 24, and the shoulder portions 24b of the breast brace 24 hold down shoulders of the infant 5 downward or toward the seat base 27. For better comfort for the infant 4, foam 21 is used on the interfacing surface of the breast brace 24.

Foam 21 may be made of suitable material such as Ethyl Vinyl Acetate (or EVA) or similar safe and soft material.

In order to effectively manage movements of the infant's legs, the mirror handle 28 also function as a leg dividing member to open legs, when seated, and to individually restrict movement of each leg.

The mirror handle 28 and the breast brace 24 are tilted forward to allow the infant 5 to lean forward, and to rest the upper body thereon to provide a stable support to maintain the bending position as shown in FIG. 12. The angle of the mirror handle 28 and the breast brace 24 may be depending on the depth and shape of the dip/pocket 27d on the seat foam 26 and seat base 27.

Lock 23 is provided with a release handle/lock handle 25. The breast brace 24 telescopically and slidably engages with the mirror-handle 28. The lock handle 25 releasably engages to lock the breast brace 24 at the desirable height. The lock handle 25 may function as height adjusting lever for actuating means for adjusting the height of the breast brace 24 as shown in FIG. 13a.

In order for better pelvis/hip immobilization, the support system 200 further comprises a lateral body immobilizer 31, allowing the seat base 27 to be adjustable to the size of child to fix the position of the lower body including gross positioning of the lumbar spine in the both the frontal (coronal) and longitudinal (sagittal) planes of motion.

For example, referring to FIGS. 10 and 14, the lateral body immobilizer 31 comprises an actuator (not shown) actuated by handles for seat adjuster 22, which are provided at the sides of the seat base 27. Movable jaw/pad (not shown) is provided under the seat foam 26, engaging/interfacing a side of the hip of the infant 5, to immobilize the hip of the infant 5. The handles 22 may be operable, i.e., moved upwardly or downwardly to actuate the movable jaw/pad to adjust the compression of the side walls 29 of the seat foam 26 toward the side of the hip of the infant 5, In particular, the side walls 29 of the seat foam 26 are configured to engage approximately with anterior superior iliac spine of the hip and greater trochanter of the infant 5 to promote the pelvis of the infant 5 be stationary. The handles 22 as shown in this embodiment may be replaced with other means/structures to provide the same functionality to immobilize the hip of the infant 5.

The height of the breast brace 24 is adjustable for shoulder immobilization according to the size or height of child/infant 5. For example, the shoulder-interfacing portions 24b of the breast brace 24 engage with the shoulders of the infant 5 to fix the position of the upper body throughout all planes of motion. The portion of the breast brace 24 that engages with the mirror handle portion 28 of the seat base 27 is provided with a plurality of means that engages with the lock 23 along its length, such that the height of the breast brace 24 in reference to the seat base 27 is releasably adjustable using the lock 23.

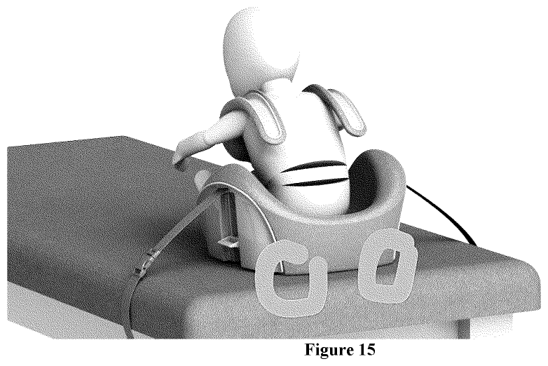

The support system 100 or 200 may further include means for anchoring (anchoring member) itself to a flat surface. For example, referring to FIG. 15, the support system 200 includes a slot for receiving an anchoring belt for anchoring it on a top of an operating bed/table.

It is to be understood that the embodiments and variations shown and described herein are merely illustrations of the principles of this invention and that various modifications may be implemented by those skilled in the art without departing from the spirit and scope of the invention.

1. A device for positioning an infant for a lumbar puncture, comprising:
a seat base defining a leg support portion, a hip support portion and a back support portion,
wherein the hip support and the back support portion receives a hip of the infant, and
the leg support portion positioned higher than the hip support position, causing thighs and knees of the infant to be close to or to touch an upper body of the infant to expose a back of the infant for the lumbar puncture.

2. The device according to claim 1 further comprising a leg dividing member for managing a movement of each leg of the infant.

3. The device according to claim 1 further comprising a body immobilizer for immobilizing a bending body position of the infant.

4. The device according to claim 3, wherein the body immobilizer comprises a height adjustable horizontal bar that holds thighs of the infant against the seat base and promote the upper body of the infant bending forward over the height adjustable horizontal bar.

5. The device according to claim 3, wherein the body immobilizer comprises a height adjustable breast brace for supporting the upper body of the infant.

6. The device according to claim 5, wherein the height adjustable breast brace is tilted forward.

7. The device according to claim 3, wherein the height adjustable breast brace comprises shoulder portions to hold down the shoulder toward the seat base to immobilize the body of the infant.

8. The device according to claim 7, wherein the height adjustable breast brace defines a chin rest portion for receiving a chin of the infant.

9. The device according to claim 1 further comprising a lateral body immobilizer adjustably and operably disposed on a lateral wall of the seat base to immobilize the hip of the infant.

10. The device according to claim 9, wherein the lateral body immobilizer comprising an actuator actuating a movable jaw to engage with a side of the hip of the infant.

11. The device according to claim 10, wherein the actuator comprising a screw member, and a handle at one end of the screw member, wherein the lateral wall of the seat base defines a port that receives the screw member for adjusting a distance from the lateral wall to suitably engage the movable jaw to the side of the hip of the infant.

12. The device according to claim 1 further comprising an anchoring member for anchoring the device on a surface of an operating table.
